(19) United States
(12) Patent Application Publication
(10) Pub. No.: US 2002/0114829 A1
(43) Pub. Date: Aug. 22, 2002

(54) Materials and methods for making improved liposome compositions

(76) Inventors: Hayat Onyuksel, Western Springs, IL (US); Israel Rubinstein, Highland Park, IL (US)

Correspondence Address: MARSHALL, GERSTEIN & BORUN, 6300 SEARS TOWER, 233 SOUTH WACKER, CHICAGO, IL, 60606-6357, US

(21) Appl. No.: 09/995,263

(22) Filed: Nov. 27, 2001

Related U.S. Application Data
(63) Continuation-in-part of application No. 09/630,699, filed on Aug. 1, 2000, now Pat. No. 6,348,214.
Application No. 09/630,699 is a division of application No. 09/155,368, filed on Dec. 18, 1998, now Pat. No. 6,197,333.

(51) Int. Cl. A61K 38/48; A61K 9/127

(52) U.S. Cl. 424/450; 424/94.63

(57) ABSTRACT

Provided are methods for treating autism, multiple sclerosis, eneuresis, Parkinson's disease, amyotrophic lateral sclerosis, brain ischemia, stroke, Cerebral palsy sleep disorder, feeding disorder and AIDS-associated dementias, using improved biologically active liposome products comprising a biologically active amphipathic compound in association with a liposome. Methods for producing the liposome products as well as methods of using the liposome products in therapeutic and diagnostic techniques are also provided.

This application is a continuation-in-part application of U.S. Ser. No. 09/630,699, filed Aug. 1, 2000, which is a divisional application of U.S. Ser. No. 09/155,368, filed Sep. 28, 1998 and now U.S. Pat. No. 6, 197,333 which issued Mar. 6, 2001 , which claims priority of International Application No. PCT/US97/05161, filed Mar. 28, 1997, which claims priority of U.S. Provisional Application No. 60/014,363, filed Mar. 28, 1996.

### BACKGROUND OF THE INVENTION

The present invention relates generally to biologically active compounds and more specifically to compounds, peptides and proteins which are amphipathic, i.e., have both hydrophilic and hydrophobic portions. Specifically, the invention relates to improved methods for the delivery and presentation of amphipathic compounds, peptides, and proteins including analogs and fragments alone and/or conjugated to other compound in association with liposomes for both diagnostic and therapeutic uses.

Of particular interest to the present invention are the biologically active amphipathic peptides which are members of the family of peptide compounds including vasoactive intestinal peptide (VIP), growth hormone releasing factor (GRF) and IL-2. More specifically, the invention relates to improved therapeutic methods for delivering peptides in the VIP/GRF or IL-2 family of peptides to targeted tissues through use of improved liposome compositions comprising a member of the VIP/GRF or IL-2 family of peptides and biologically active analogs, fragments and modulators thereof.

VIP is a 28-amino acid neuropeptide which is known to display a broad profile of biological actions and to activate multiple signal transducing pathways. See, Said, Peptides 5 (Suppl. 1): 149-150 (1984) and Paul and Ebadi, Neurochem. Int. 23:197-214 (1993). A Schiff-Edmundson projection of VIP as a π-helix reveals segregation of apolar and polar residues onto the opposite faces of the helix and that this amphipathic character is also evident when VIP is modeled as a distorted α-helix, which is reported in Musso, et al., Biochemistry 27:8147-8181 (1988). A correlation between the helix-forming tendency of VIP analogs and their biological activity is described in Bodan et al, Bioorgan. Chem. 3:133-140 (1974). In pure water, the spectral characteristics of VIP are consistent with those of a random coil. However, organic solvents and anionic lipids induce helical-information in the molecule. See, Robinson et al, Biopolymers 21:1217-1228 (1983); Hamed, et al., Biopolymers 22:1003-1021 (1983); and Bodanszky, et al., Bioorganic Chem. 3:133-140 (1974).

Short peptides capable of forming amphipathic helices are known to bind and penetrate lipid bilayers. See, Kaiser and Kezdy, Ann. Rev. Biophys. Biophysical Chem. 15:561-581 (1987) and Sansom, Prog. Biophys. Molec. Biol. 55:139-235 (1991). Examples include model peptides like (LKKLLKL-), which are disclosed in DeGrado and Lear, J. Am. Chem. Soc. 107:7684-7689 (1985), and the 26-residue bee venom peptide, melittin, disclosed in Watata and Gwozdzinski, Chem-Biol. Interactions 82:135-149 (1992). Possible mechanisms for the binding include alignment of peptide monomers parallel to the surface of the bilayer mediated by electrostatic interactions between polar amino acids and phospholipid head groups, and insertion of peptide aggregates into the apolar bilayer core, stabilized in part, by the hydrophobic effect. See, Sansom, Prog. Biophys. Molec. Biol. 55:139-235 (1991).

VIP belongs to a family of homologous peptides, other members of which include peptide histidine isoleucine (PHI), peptide histidine methionine (PHM), growth hormone releasing factor (GRF), hypocretins, pituitary adenylate cyclase activating peptide (PACAP), secretin, and glucagon. Like VIP, the other members of the VIP/GRF family of peptides, and biologically active analogs thereof, can form amphipathic helices capable of binding lipid bilayers. The biological action of members of the VIP/GRF family of peptides are believed to be mediated by protein receptors expressed on the cell surface and intracellular receptors and it has recently been demonstrated that calmodulin is likely to be the intracellular receptor for VIP [Stallwood, et al., J. Bio. Chem. 267:19617-19621 (1992); and Stallwood, et al, FASEB J. 7:1054 (1993)].

The pleiotropic distribution of VIP is correlated with its involvement in a broad spectrum of biological activities, and growing evidence suggests that VIP plays a major role in regulating a variety of important functions in many organs. Physiological actions of VIP have been reported on the cardiovascular, respiratory, reproductive, digestive, immune, and central nervous systems, as well as metabolic , endocrine and neuroendocrine functions (for review, Said, Trends Endocrinol. Metab. 2:107-112 (1991)). In many cases, VIP acts as a neurotransmitter or neuromodulator and is released into the local circulation at small concentrations. Among the functions that VIP is believed to mediate or promote (Said, Trends Endocrinol. Metab. 2:107-112 (1991) Paul et al., Neurochem. Int. 23:197-214 (1993)), are vasodilation of cerebral, coronary, peripheral, and pulmonary blood vessels, linked to the regulation of vascular tone; the relaxation of gastrointestinal, uterine, and tracheobronchial smooth muscles; exocrine secretion, water and anions by intestinal, respiratory, and pancreatic epithelia; stimulation of the male and female activity and responses; release and regulation of neuroendocrine functions (renin release, melatonin secretion); inhibition of the immune system (inhibition of platelet aggregation); and stimulation and protection of neuronal cells.

New VIP functions such as inhibition of vascular smooth muscle cell growth, proliferation of cultured human keratinocytes, the release of neutrophic and growth factors involved in cell differentiation and ontogeny, and antioxidant properties have been recently proposed but still need additional studies (Muller et al., Mol. Neurobiol. 10:115-134 (1995); Said, Trends Endocrinol. Metab. 2:107-112 (1991)).

Some human diseases today are known to be associated with the deficiency in the release of VIP. The deficiency of VIP has been linked to the pathogenesis of several diseases, such as cystic fibrosis, diabetic impotence, congenital mengacolon in Hirschsprung's disease, and achalasia of the esophagus. Furthermore, VIP insufficiency may be a cause of bronchial hyperactivity in asthmatic airways since VIP is known to mediate airway relaxation in humans, and lung tissues of asthmatic patients showed a selective absence of VIP nerves (Ollerenshaw et al., N. Engl. J. Med. 320:1244-1248 (1989)). Finally, Avidor et al., Brain Res. 503:304-307 (1989) observed an increase in brain VIP gene expression in a rat model for spontaneous hypertension, thought to be associated with the pathophysiology of the disease.

On the other hand, the excessive release of VIP has been linked to the pathogenesis of few diseases. One of the pathological syndromes is pancreatic cholera, a watery diarrhea-hypocholaremia-hypochloridria condition (Krejs, Ann. N.Y. Acad. Sci. 527:501-507 (1988)). Certain tumors, especially pancreatic, bronchogenic, and neurogenic, have been associated with elevated circulatory levels of VIP. In addition, it has also been suggested that increased levels of neuropeptides, including VIP, are found in neonatal blood of autistic children (Nelson, et al., American Journal of Epidemiology 151 (11 Supplement):pS3 Jun. 1, 2000).

Due to the numerous physiological actions of VIP, the use of VIP as a drug has been of growing interest. The potential therapeutic developments of VIP include treatment of diseases where regional blood flow is deprived. These include hypertension by reducing systemic vascular overload, left ventricular failure, congestive heart failure, and coronary or peripheral ischemia. VIP infusion in man for 10 hours was shown to reduce total peripheral resistance by 30 % and increase forearm blood flow by 270% (Frase et al., Am. J. Cardiol. 60:1356-1361 (1987)). Moreover, Smiley, Am. J. Med. Sci. 304:319-333 (1992) showed VIP-immunoreactive nerves in the skin and plasma levels of VIP were found to be low in patients with schleroderma, thus treatment with VIP may restore this impaired response. Other diseases which could be treated by administration of VIP include treatment of asthmatic bronchospasm. VIP has been shown to protect against bronchoconstriction in asthmatic patients and as a relaxant of tracheobronchial smooth muscle (Morice et al., Lancet 26 2(8361):1225-1227 (1983)). Its anti-inflammatory properties could further enhance its therapeutic value in asthma (Said, Biomed. Res. 13 (Suppl. 2):257-262 (1992)). Administration of VIP could also be used in -the prevention and/or reduction of tissue injury. The peptide has been described to prevent neuronal cell death produced by the external envelope protein gp120 of the human immunodeficiency virus in vitro (Gozes et al., Mol. Neurobiol. 3:201-236 (1989); Hökfelt, Neuron. 7:867-879 (1991)), which may lead to a potential therapy for AIDS dementia as well as treatment of Alzheimer's disease. Likewise, the acute inflammatory lung injury induced by a variety of insults including oxidant stress was diminished by the presence of VIP (Berisha et al., Am. J. Physiol. 259:L151-L155 (1990)). VIP added to certain pneumoplegic solutions was also shown to improve rat lung preservation before transplantation (Alessandrini et al., Transplantation 56:964-973 (1993)).

A major factor limiting in vivo administration of VIP has been its reduced bioavailability at target tissues mostly because of proteolytic degradation, hydrolysis, and/or a multiplicity of conformations adopted by the peptide. It has been speculated that intracellular delivery of VIP alone and/or VIP-calmodulin mixtures could bypass the requirement for cell-surface binding of the peptide and thus enhance the biological actions of the peptide. Provision of the peptides expressed in and on liposomes would possibly permit intracellular delivery, since lipid bilayers of liposomes are known to fuse with the plasma membrane of cells and deliver entrapped contents into the intracellular compartment.

Characterization of the structure and properties of liposomes led to many proposed uses for the vesicle as vehicles to effect targeted drug delivery, most of which failed to materialize for any of a number of various reasons. Most prominently, the therapeutic parenteral use of conventional liposomes was found to be limited because of rapid uptake into the reticuloendothelial system by mononuclear phagocytic cells [Gregoriadias and Ryman, Eur. J. Biochem. 27:485-491 (1972); Beaumier, and Hwang, Biochem. Biophys. Acta 731:23-30(1983)]. Uptake by this particular cell type is advantageous under the limited conditions wherein the targeted cell or tissue itself is part of the reticuloendothelial system, but uptake by phagocytic cells generally leads to degradation of compounds to be delivered, thereby posing a serious drawback to delivering a compound to other cell or tissue types.

In attempts to overcome problems inherent to liposome drug delivery, research turned to several approaches including identification of compounds which would be released back into the blood following liposome uptake by the reticuloendothelial system, alternatives to intravenous liposome administration, and use of various compounds, for example, cholesterol, to increase liposome stability in the bloodstream [Kirby, et al., Biochem. J. 186-591-598 (1980); Hwang, in Liposomes from biophysics to therapeutics, Ostro (ed.) Marcel Decker: New York (1987) pp. 109-156; Beaumier, et al., Res. Comm. Chem. Pathol. Pharmacol. 39:227-232 (1983)]. Still other investigations examined various lipid compositions to form the liposome bilayer which more closely mimic the naturally occurring bilayer of red blood cell. Such efforts led to increased liposome half-life in circulation [Allen and Chonn, FEBS Lett. 223:42-46 (1987); Gabizon and Papahadjopoulos, Proc. Natl. Acad. Sci. (USA) 85:6949-6953 (1988)].

PCT Publication WO 95/27496 and Gao, et al., Life Science 54:247-252 (1994) describe the use of liposomes for delivery of VIP in comparison to its delivery in aqueous solution. Encapsulation of VIP in liposomes was found to protect the peptide from proteolytic degradation and to significantly enhance the ability of VIP and to effect a decrease in mean arterial pressure in comparison to VIP in aqueous solution in hypertensive hamsters. Liposome-associated VIP was found to significantly decrease mean arterial blood pressure for a period of approximately 12 minutes, with lowest blood pressure observed almost 5 minutes after initial administration. The publication also demonstrated binding of VIP in aqueous solution to liposomes and penetration of the peptide into the liposome bilayer. It was speculated that binding of VIP to liposomes might prevent loss of peptide activity either by partitioning of the peptide into the liposome membrane, stabilizing the peptide against proteolysis, or restricting the peptide in a biologically active conformation. Whatever the reason, encapsulation of VIP in liposomes enhanced in vivo biological activity of the peptide by both prolonging the effect and increasing the magnitude of the effect in lowering blood pressure of hypertensive hamsters. Nevertheless, there remains a desire in the art to provide further improvements in the therapeutic and diagnostic delivery of biologically active peptides such as VIP.

Of interest to the present invention is the observation of increased half-life of circulating protein through conjugation of the protein to a water soluble polymer [Nucci, et al., Adv. Drug Del. Rev. 6:133-151 (1991); Woodle, et al., Proc. Intern. Symp. Control. Rel. Bioact. Mater. 17:77-78 (1990)]. This observation led to the development of sterically stabilized liposomes (SSL) (also known as “PEG-liposomes”) as an improved drug delivery system which has significantly minimized the occurrence of rapid clearance of liposomes from circulation. [Lasic and Martin, Stealth Liposomes, CRC Press, Inc., Boca Raton, Fla. (1995)]. SSL are polymer-coated liposomes, wherein the polymer, preferably polyethylene glycol (PEG), is covalently conjugated to one of the phospholipids and provides a hydrophilic cloud outside the vesicle bilayer. This steric barrier delays the recognition by opsonins, allowing SSL to remain in circulation much longer than conventional liposomes [Lasic and Martin, Stealth Liposomes, CRC Press, Inc., Boca Raton, Fla. (1995); Woodle, et al., Biochem. Biophys. Acta 1105:193-200 (1992); Litzinger, et al., Biochem. Biophys. Acta 1190:99-107 (1994); Bedu Addo, et al., Pharm. Res. 13-718-724 (1996)] and increases the pharmacological efficacy of encapsulated agents, as demonstrated for some chemotherapeutic and anti-infectious drugs [Lasic and Martin, Stealth Liposomes, CRC Press, Inc., Boca Raton, Fla. (1995)]. Studies in this area have demonstrated that different factors affect circulation half-life of SSL, and ideally, the mean vesicle diameter should be under 200 nm, with PEG at a molecular weight of approximately 2,000 Da at a concentration of 5% (9-12) [Lasic and Martin, Stealth Liposomes, CRC Press, Inc., Boca Raton, Fla. (1995); Woodle, et al., Biochem. Biophys. Acta 1105:193-200 (1992); Litzinger, et al., Biochem. Biophys. Acta 1 190:99-107 (1994); Bedu Addo, et al., Pharm. Res. 13:718-724 (1996)]. Preparation of SSL having these physical properties and including a bioactive compound, however, is not without complications as activity of the associated compound can be lost in preparation of SSL having desirable characteristics. This is particularly the case where an extrusion process is used to obtain small size liposomes with a narrow particle size distribution. For reasons which are not completely understood, such extrusion methods substantially reduce the biological activity peptide components associated with the liposomes. Accordingly, there remains a desire for improved liposome compositions which are sterically stable but which maintain the biological activity of associated peptide agents.

Also of interest to the present invention is the disclosure of PCT Publication WO 93/120802 which relates to multilamellar liposomes useful for enhancement of organ imaging with acoustics (ultrasound). The publication describes various liposome compositions ranging in size from 0.8 to 10 microns including a tissue specific ligand, such as an antibody, antibody fragment or a drug incorporated into the lipid bilayer, in order to facilitate tissue specific targeting. The oligolamellar liposomes are prepared by processes such as lyophilization, repeated freeze-thaw, or modified double emulsion techniques to produce internally separated bilayers. Preferred liposomes are said to range from 1.0 to 3.0 microns in diameter. It has thus far been more difficult to produce liposomes which are readily detectable by conventional ultrasound techniques less than about 0.5 microns in size. Accordingly, there remains a desire for improved liposome compositions which may be efficiently produced and which have average particle sizes less than about 0.5 microns. Moreover, there remains a desire for improved liposome compositions which are efficiently produced, stable in vivo, and provide a higher degree of resolution upon acoustic imaging.

Thus, there exists a need in the art to provide further improvements in the use of liposome technology for the therapeutic and diagnostic administration of bioactive molecules. More specifically, there remains a desire in the art for improved methods for administration of amphipathic peptides including, but not limited to, members of the VIP/GRF family of peptides in liposomes in order to achieve a more prolonged and effective therapeutic effect.

### SUMMARY OF THE INVENTION

The present invention provides methods of treating a variety of disease states using liposome compositions prepared as described in U.S. patent application Ser. No. 6, 197,333, issued Mar. 6, 2001, and PCT Publication No. WO 97/35561, published Oct. 2, 1997, both of which are incorporated herein by reference in their entireties. Methods of the invention provide therapeutic treatment for disease states as described herein. The liposomal formulations of the invention deliver and enhance bioactivity of the biologically active compounds, peptides, and proteins, including analogs and fragments thereof, alone and/or conjugated to other compounds in a manner which provides improvements in the efficacy and duration of the biological effects of the associated peptides. Increased efficacy and duration of the biological effect is believed to result, at least in part, from interaction of the compound with the liposome in such a manner that the compound attains, and is maintained in, an active or more active conformation than the compound in an aqueous environment. The invention thus overcomes the problems associated with previous liposomal formulations, such as, but not limited to, uptake by the reticuloendothelial system, degradation of the compound, or delivery of the compound in an inactive conformation. Particularly preferred amphipathic compounds useful with the invention include any member of the vasoactive intestinal peptide (VIP)/growth hormone releasing factor (GRF) or IL-2 family of peptides which includes biologically active analogs thereof. The mammalian and non-mammalian VIP/GRF family of peptides includes functional analogs of VIP and GRF, peptide histidine isoleucine (PHI), peptide histidine methionine (PHM), growth hormone releasing factor (GRF), hypocretins, pituitary adenylate cyclase activating peptide (PACAP), secretin, and glucagon. Preferred methods of the invention utilize liposome compositions comprising a member of the VIP/glucagon/secretin family of peptides including peptide fragments and analogs. The biologically active peptide products of the invention may be utilized in a wide variety of therapeutic and diagnostic uses wherein it is desired to deliver a high level of biologically active compound or to , detect targeted delivery of the liposome product as will be described below.

In one aspect, the invention provides methods of treating a disease state selected from the group consisting of autism, multiple sclerosis, eneuresis, Parkinson's disease, amyotrophic lateral sclerosis, brain ischemia, stroke, cerebral palsy (CP) sleep disorder, feeding disorder and AIDS-associated dementias, comprising the step of administering to an individual suffering from the disease state an amount of a liposome composition effective to alleviate conditions associated with the disease state, said liposome composition prepared by a method comprising the steps of: a) mixing a combination of lipids wherein said combination includes at least one lipid component covalently bonded to a water-soluble polymer; b) forming sterically stabilized liposomes from said combination of lipids; c) obtaining liposomes having an average diameter of less than about 300 nm; and d) incubating liposomes from step (c) with a biologically active amphipathic compound under conditions in which said compound becomes associated with said liposomes from step (c) in an active conformation, wherein at least one amphipathic compound is a member of the VIP/glucagon/secretin family of peptides including peptide fragments and analogs. In one embodiment, methods of the invention employ active liposome compositions which comprise unilamellar liposomes. In another embodiment, these liposome compositions are multivesicular liposomes. In aspects of the invention wherein the liposome compositions are multivesicular liposomes, methods are provided wherein the liposome compositions produced by carrying out the steps of sequentially dehydrating and rehydrating liposomes obtained in step (c) with said biologically active peptide.

Preferably, methods utilize liposome compositions wherein the water soluble polymer is polyethylene glycol (PEG). Also preferred are methods wherein the amphipathic compound is characterized by having one or more α- or π-helical domains in its biologically active conformation. In more preferred methods, the compound is a member of the vasoactive intestinal peptide (VIP)/growth hormone releasing factor (GRF) family of peptides. In another aspect, the compound is a member of the VIP/glucagon/secretin family of peptides, including peptide fragments and analogs thereof.

Methods of the invention include those wherein liposomes obtained in step (c) have an average diameter or less than about 200 nm. In a preferred aspect, the liposomes obtained in step (c) have an average diameter or less than about 100 nm. In one aspect, the liposomes are obtained in step (c) by extrusion to form liposomes having a selected average diameter. Alternatively, methods employ liposome which are obtained in step (c) by size selection.

In one aspect, methods of the invention utilize liposome which are formed from a combination of lipids that consists of distearoyl-phosphatidylethanolamine covalently bonded to PEG (PEG-DSPE), phosphatidylcholine (PC), and phosphatidylglycerol (PG) in further combination cholesterol (Chol). In a preferred method, these lipids are combined with cholesterol in a PEG-DSPE:PC:PG:Chol molar ratio of 0.5:5:1:3.5.

### DETAILED DESCRIPTION OF THE INVENTION

The present invention provides improved methods of preparing biologically active liposome products comprising biologically active amphipathic compounds in association with a liposome. The preferred amphipathic compounds are characterized by having hydrophilic and hydrophobic domains segregated to the extent that the hydrophobic domain is capable of associating with or within the liposome bilayer. Compounds of the invention preferably attain a biologically active conformation in association with or within the liposome bilayer. Active conformations are those in which the desired compound is most likely to be capable of effecting its normal biological activity, for example, through receptor or ligand recognition and binding. Compounds of the invention may be characterized by having one or more discrete α- or π-helical domains which segregate the hydrophobic and hydrophilic domains. Preferred compounds of the invention are members of the VIP/GRF or IL-2 peptide family. The most preferred compound of the invention is a member of the VIP/glucagon/secretin or IL-2 family of peptides including peptide fragments and analogs. While biologically active compounds are associated with the liposome bilayer, the association is not irreversible and the compound may be released either quickly or over time from association with the liposome, depending on properties of the liposome and the compound.

In contrast to prior art methods which frequently include the step of extruding peptide-containing liposomes through membranes and filters to obtain liposomes of a desired size, the liposomes according to the present invention are obtained having a diameter of less than 300 nm prior to being contacted with the active compound ingredient. Liposomes of this size may be obtained using an extrusion step which modifies liposomes, thereby reducing the size of the liposomes to a preferred average diameter prior to being incubated with the biologically active compound. Alternatively, liposomes of the desired size may be selected using techniques such as filtration or other size selection techniques. While the size-selected liposomes of the invention should have an average diameter of less than about 300 nm, it is preferred that they are selected to have an average diameter of less than about 200 nm with an average diameter of less than about 100 nm being particularly preferred. When the biologically active liposome product is a unilamellar liposome, it preferably is selected to have an average diameter of less than about 200 nm. The most preferred unilamellar liposomes of the invention have an average diameter of less than about 100 nm. It is understood, however, that multivesicular liposomes of the invention derived from smaller unilamellar liposomes will generally be larger and may have an average diameter of about less than 1000 nm. Preferred multivesicular liposomes of the invention have an average diameter of less than about 800 nm, and less than about 500 nm while most preferred multivesicular liposomes of the invention have an average diameter of less than about 300 nm.

Liposomes according to the invention may be produced from combinations of lipid materials well known and routinely utilized in the art to produce liposomes and including at least one lipid component covalently bonded to a water-soluble polymer. Lipids may include relatively rigid varieties, such as sphingomyelin, or fluid types, such as phospholipids having unsaturated acyl chains. Polymers of the invention may include any compounds known and routinely utilized in the art of SSL technology and technologies which are useful for increasing circulatory half-life for proteins, including for example polyvinyl alcohol, polylactic acid, polyglycolic acid, polyvinylpyrrolidone, polyacrylamide, polyglycerol, polyaxozlines, or synthetic lipids with polymeric headgroups. The most preferred polymer of the invention is PEG at a molecular weight between I 000 and 5000. Preferred lipids for producing liposomes according to the invention include distearoyl-phosphatidylethanolamine covalently bonded to PEG (PEG-DSPE), phosphatidylcholine (PC), and phosphatidylglycerol (PG) in further combination with cholesterol (Chol). According to a preferred embodiment of the invention, a combination of lipids and cholesterol for producing the liposomes of the invention comprise a PEG-DSPE:PC:PG:Chol molar ratio of 0.5:5:1:3.5.

The liposomes produced according to the methods of the invention are characterized by improved stability and biological activity and are useful in a variety of therapeutic, diagnostic and/or cosmetic applications. According to one embodiment, the invention comprehends a composition comprising a biologically active liposome product wherein said biologically active amphipathic compound has anti-oxidant activity, anti-aging, anti-wrinkle formation or wound healing capacity. Compositions of this type may be of cosmetic or therapeutic nature. The preferred cosmetic composition includes a biologically active member of the VIP/glucagon/secretin or IL-2 family of peptides including peptide fragments and analogs. The invention also provides an oral controlled release preparation for the treatment of a gastrointestinal disorder wherein said preparative method further comprises the step of encapsulating the biologically active liposome product in an enteric coating. The oral controlled release preparation is useful in a variety of gastrointestinal disorders including those selected from the group consisting of inflammatory bowel disorder, chronic constipation, Hirschprung's disease, achalasia, infantile hypertrophic pyloric stenosis, and ulcers. The preferred oral preparation includes a biologically active member of the VIP/glucagon/secretin or IL-2 family of peptides including peptide fragments and analogs. Liposome preparations comprising a biologically active member of the VIP/glucagon/secretin or IL-2 family of peptides including peptide fragments and analogs are also a promising therapeutic agent for conditions such as asthma, systemic and pulmonary hypertension, scleroderma, myocardial ischemia, impotence and baldness. The invention further provides methods for preserving a bodily organ, tissue, or cell type for storage and transplantation in a recipient comprising the step of incubating said organ in a liposome composition comprising a member of the VIP/glucagon/secretin or IL-2 family of peptides including peptide fragments and analogs.

The invention further provides methods of treating autism, multiple sclerosis, eneuresis, Parkinson's disease, amyotrophic lateral sclerosis, brain ischemia, stroke, CP sleep disorder, feeding disorder and AIDS-associated dementia by administering a amount of a composition of the invention effective to ameliorate pathological conditions associated with autism, multiple sclerosis, eneuresis, Parkinson's disease, amyotrophic lateral sclerosis, brain ischemia, stroke, CP sleep disorder, feeding disorder and AIDS-associated dementias

The invention further provides methods of administering a biologically active amphipathic compound to a target tissue comprising the steps of: preparing a biologically active liposome product comprising a biologically active amphipathic compound in association with a liposome according to the methods of the invention and administering a therapeutically effective amount of the liposome product to said target tissue. The liposome products of the invention may be administered intravenously, intraarterially, intranasally such as by aerosol administration, nebulization, inhalation, or insufflation, intratracheally, intra-articularly, orally, transdermally, subcutaneously, topically onto mucous membranes, such as, but not limited to, oral mucosa, lower gastrointestinal mucosa and conjunctiva, and directly onto target tissues.

An exemplary regiment in the treatment, for example, of autism, multiple sclerosis, eneuresis, Parkinson's disease, amyotrophic lateral sclerosis, brain ischemia, stroke, CP sleep disorder, feeding disorder and AIDS-associated dementias, would include administration of from 0.001 mg/kg body weight to about 1000 mg/kg, from about 0.01 mg/kg to about 100 mg/kg, from about 0.1 mg/kg to about 100 mg/kg, about 1.0 mg/kg to about 50 mg/kg, or from about 1 mg/kg to about 20 mg/kg, given in daily doses or in equivalent doses at longer or shorter intervals, e.g., every other day, twice weekly, weekly, monthly, semi-annually, or even twice or three times daily. Alternatively, dosages may be measured in international units ( IU) ranging from about 0.001 IU/kg body weight to about 1000 IU/kg, from about 0.01 IU/kg to about 100 IU/kg, from about 0.1 IU/kg to about 100 IU /kg, from about 1 IU/kg to about 100 IU/kg, from about 1 IU/kg to about 50 IU/kg, or from about 1 IU/kg to about 20 IU/kg. Administration may be oral, intravenous, subcutaneous, intranasal, inhalation, transdermal, transmucosal, or by any other route discussed herein.

Biologically active compounds in therapeutic methods can be administered at significantly reduced dosage levels as compared to administration of the compound alone, particularly wherein the compound has a particularly short half life or lowered bioactivity in circulation. For example, VIP in association with SSL can be expected to exhibit enhanced and prolonged bioactivity in comparison to VIP administered alone. Generally, the biologically effective amount of VIP in SSL is about 50 to 75 percent less by weight than the biologically effective amount of VIP in aqueous solution. Regardless of which bioactive compound is associated with SSL, the liposome product must be tested in order to determine a biologically effective amount required to achieve the same result effected by the compound administered by conventionally means. The worker of ordinary skill in the art would realize that the biologically effective amount of a particular compound when delivered by conventional means would serve as a starting point in the determination of an effective amount of the compound in SSL. It would therefore be highly predictive that the same and lesser dosages in SSL would be effective as well and merely routine to determine the minimum dosage required to achieve a desired biological effect. In the case of VIP administration, for example, if conventional administration would require a dosage of 20 mg, VIP in SSL would likely require 5 to 10 mg in order to achieve the same effect. Typically, a biologically effective amount of intravenously administered VIP would total 0.01 to 50 mg daily or 0.1 to 500 mg VIP in capsule form.

Association of a biologically active compound with SSL of the invention would be expected to increase the magnitude of the biological effects of the compound from about 50 to 100% over the effects observed following administration of the compound alone. Likewise, association with SSL of the invention would be expected to invoke a longer lasting biological effect.

The invention further provides improved diagnostic compositions comprising multivesicular biologically active liposome products and methods for their use comprising the steps of: preparing a biologically active liposome product comprising a biologically active amphipathic compound in association with a multilamellar liposome prepared according to the methods of the invention; administering a diagnostically effective amount of the liposome product to a target tissue; and detecting uptake or interaction of the liposome product at the target tissue. According to one aspect of the invention, the target tissue is a tumor. In one aspect of the method, the liposome product is detectably labeled with a label selected from the group including a radioactive label, a fluorescent label, a non-fluorescent label, a dye, or a compound which enhances magnetic resonance imaging (MRI). According to the preferred embodiment of the invention, the liposome product is detected by acoustic reflectivity. Diagnostic liposome products for detection by acoustic imaging generally have an average diameter of less than about 1000 nm, but preferably, the diagnostic liposome products have an average diameter of less than 600 nm and most preferably have an average diameter of less than about 300 nm.

The invention also provides use of a biologically active liposome product comprising a biologically active amphipathic compound and produced according to methods of the invention for the treatment of inflammation, chronic obstruction pulmonary disease, increased secretion of mucin, acute food impaction, rhinitis, Kartagener's syndrome, cystic fibrosis, bronchiectasis, hypertension, allergy, Alzheimer's disease, cerebral palsy, stroke, atherosclerosis, inflammatory bowel disorder, chronic constipation, Hirschprung's disease, achalasia, infantile hypertrophic pyloric stenosis, ulcers, to enhance or decrease cell proliferation, prevent apoptosis, to promote wound healing in a body organ or tissue, and to prevent cell, organ and tissue rejection, autism, multiple sclerosis, eneuresis, Parkinson's disease, amyotrophic lateral sclerosis, brain ischemia, stroke, cerebral palsy (CP) sleep disorder, feeding disorder and AIDS-associated dementias, impotence and female arousal sexual dysfunction. As discussed herein, neonatal blood from autistic children has been shown to have increased levels of neuropeptides, including a member of the VIP/glucagon/secretin or IL-2 family of peptides including peptide fragments and analogs. One possible explanation for this observation is that an endogenously expressed member of the VIP/glucagon/secretin or IL-2 family of peptides including peptide fragments and analogs may be biologically inactive (or partially inactivated). Because the circulating peptide is inactive, and its effects not realized, additional peptide is continually produced to achieve the desired effect. Administration of a member of the VIP/glucagon/secretin family of peptides including peptide fragments and analogs in a composition of the invention, which maintains a member of the VIP/glucagon/secretin or IL-2 family of peptides including peptide fragments and analogs in a biologically active conformation, would be expected to actuate biological processes dependent on a member of the VIP/glucagon/secretin or IL-2 family of peptides including peptide fragments and analogs that the endogenous inactive peptide cannot. As an alternative explanation, VIP receptors are rendered dysfunctional to the extent that native VIP cannot interact, whereas VIP in a micelle composition of the invention is able to either recognize and interact with the modified receptor, or able to effect its biological activity through a non-receptor mediated pathway.

U.S. Pat. No. 6,197,333, the disclosure of which is hereby incorporated described results described results of use of VIP associated liposomes according to the invention. Specifically, VIP-PEG-liposomes were prepared as follows. DSPE linked to PEG (molecular weight 1,900), PG, PC, and cholesterol (molar ration 0.5:1:5:3.5) were dissolved in chloroform in a round bottom flask. The solution was dried overnight in a rotoevaporator and the resulting film desiccated overnight. The lipid film was rehydrated with saline, pH 6-7, while vortexing, and then sonicated for at least 5 minutes. The liposome preparation thus formed was extruded through stacked Nucleopore filters with pore sizes 200nm, 100 nm, and 50 nm, respectively, until the mean size of PEG-liposome was 80-100 nm as determined by quasi elastic light scattering. VIP and trehalose, a cryoprotectant, were added to the extruded liposome preparation in polypropylene tubes, the mixture snap-freezed in ethanol- or acetone-dry ice bath for at least 20 minutes, and lyophilized overnight under similar conditions. Free VIP was separated from VIP-PEG-liposomes using Bio Gel A-5m column chromatography. The size of the PEG-liposomes in original solution and VIP-PEG-liposomes was determined by quasi elastic light scattering. Lipid concentration in PEG-liposomes in the original solution and in VIP-PEG-liposomes was determined by inorganic phosphate assay. VIP concentration in VIP-PEG-liposomes was determined by an ELISA assay.

To determine VIP concentration in VIP-PEG-liposomes, 1% sodium dodecyl sulfate, a detergent, was added to an aliquot of the VIP-PEG-liposome preparation to release associated VIP before assay. PEG-liposome and 1% sodium dodecyl sulfate alone did not interfere with the ELISA assay. Non-limiting examples from preliminary experiments using these preparations indicated increased and prolonged biological potency to target tissues of mammals as described below.

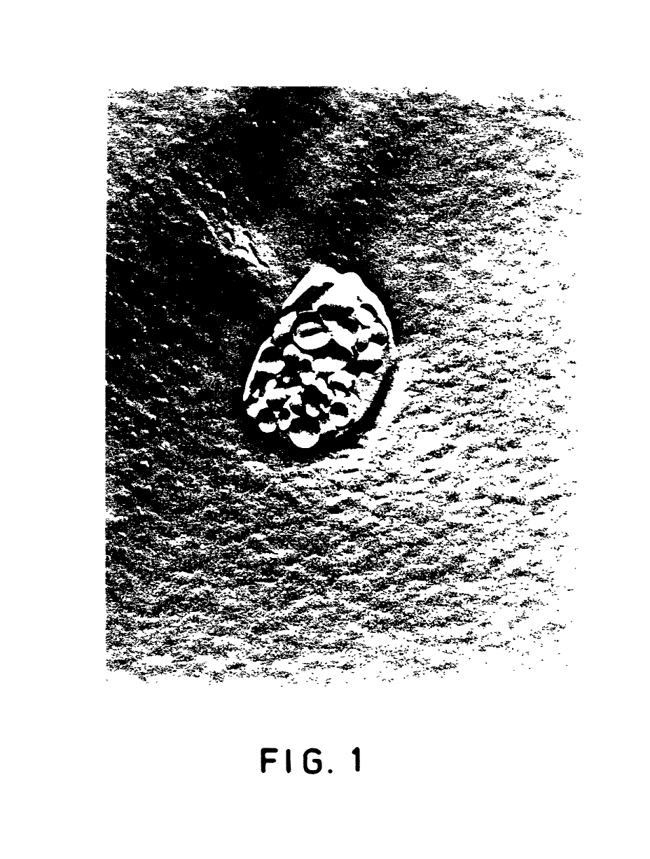

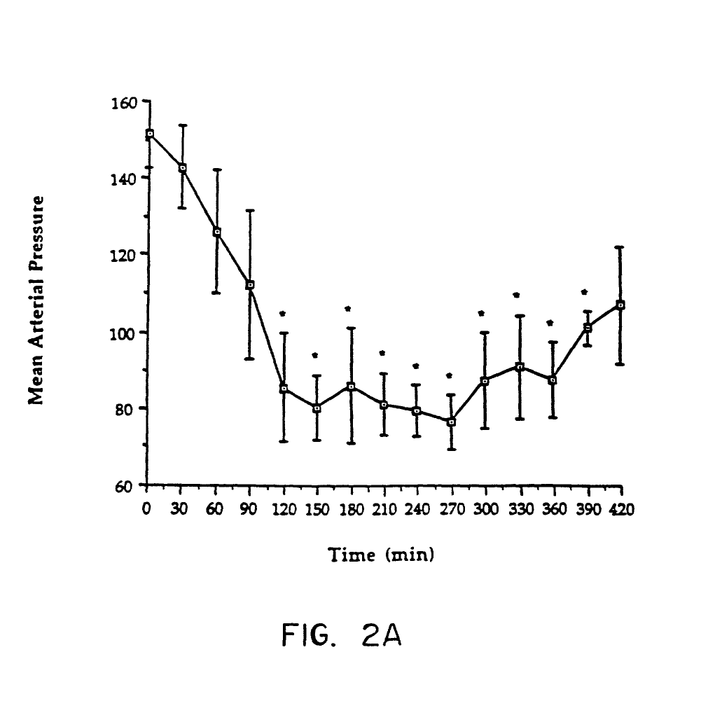

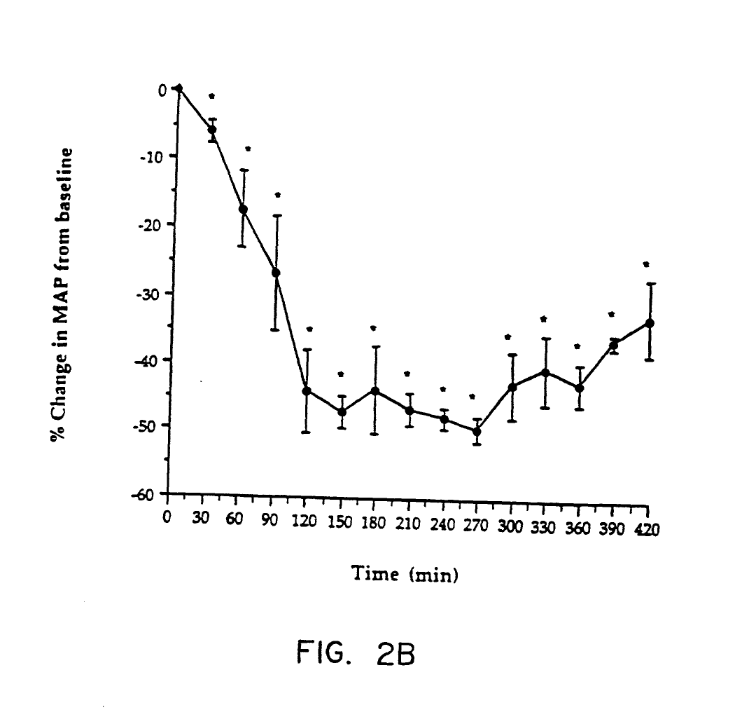

U.S. Pat. No. 6,197,333 further disclosed that a bolus intravenous injection of 1.0 nmol VIP-PEG-liposome compound acted to decrease mean arterial pressure (MAP) in hamsters with spontaneous hypertension. The results are reproduced herein as FIGS. 2A and 2B; FIG. 2A showing the actual decrease on arterial pressure and FIG. 2B showing the percent change. Data are mean values ± one standard error of the mean; an asterisk indicates statistically significant values compared to control with p value less than 0.05. Results indicated a significant, gradual and sustained decrease in mean arterial pressure reaching a nadir within 2 hours after injection of VIP-PEG-liposomes which lasted throughout the observation period of 7 hours.

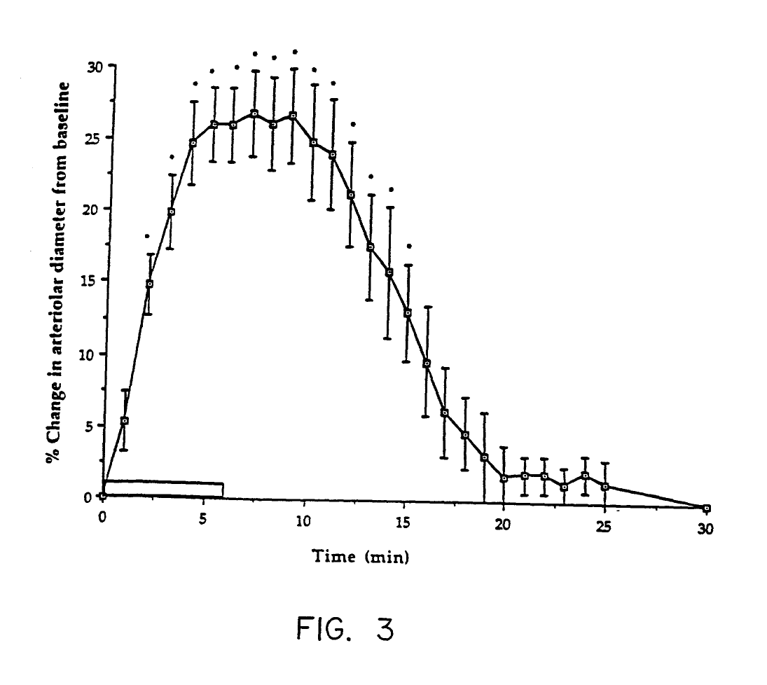

According to another experiment, normotensive hamsters were suffused onto the cheek pouch for 7 minutes with 0.1 nmol VIP-PEG-liposome composition which produced a significant increase in mean arterial diameter in situ. The results of this experiment are shown in FIG. 3 with data and significance indicated for results in FIGS. 2A and 2B above. A significant increase in arteriolar diameter from baseline was observed with maximal effect within 5 minutes from the start of suffusion. Arterial diameter returned to baseline 9 minutes after suffusion was discontinued.

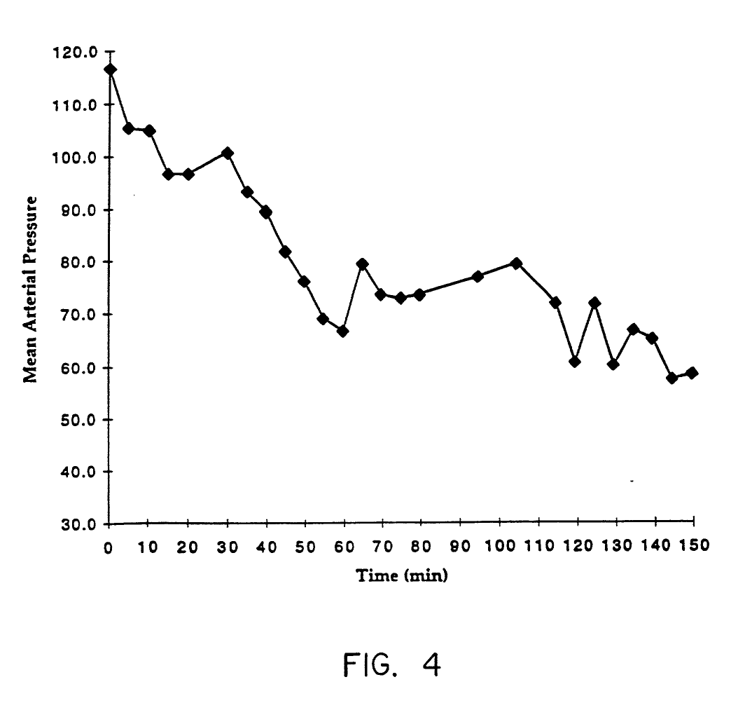

In still another experiment, 1.0 nmol VIP-PEG-liposome composition was superfused for 30 minutes into the nostril of a hypertensive hamster which resulted in a decrease in arterial pressure that persisted at least 150 minutes. These results are shown in FIG. 4. A gradual and sustained decrease in mean arterial pressure to the normal range was detected that lasted throughout the observation period of 2.5 hours.

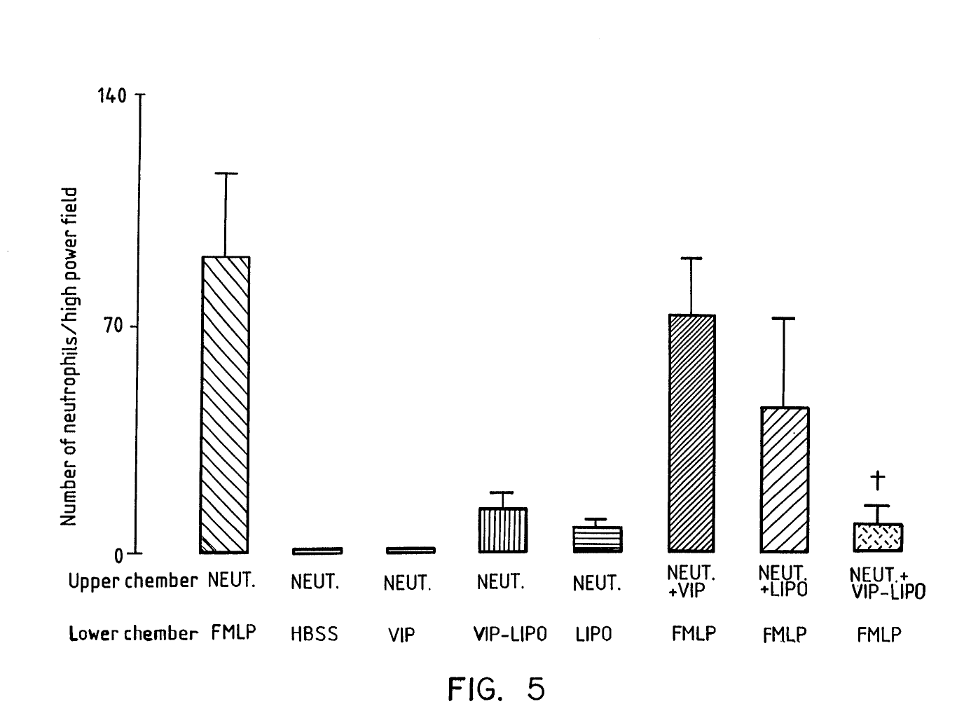

Finally, as another experiment the effect of VIP-PEG-liposomes on neutrophil chemotaxis was examined using a two chamber apparatus routinely employed for in vitro analysis of chemotaxis. The results of the experiment are shown in FIG. 5. Neutrophil migration from the upper chamber into the lower chamber in response to formyl-methionyl-leucyl-phenylalanyl (fmlp) peptide in the lower chamber was initially established at a baseline control. Neutrophil migration against media (Hank's balanced salt solution, HBSS) and VIP alone in the lower chamber was shown to be negligible, and minor levels of neutrophil migration were detected against VIP-PEG-liposomes and PEG-liposome in the lower chamber. When neutrophils and VIP were added together in the upper chamber, significant migration was observed against fmlp in the lower chamber, with slightly lower levels of cell migration observed against fmlp with neutrophils and PEG-liposomes together in the upper chamber. Finally, neutrophil migration against fmlp was reduced to almost negligible levels when VIP-PEG-liposomes were added with the cells in the upper chamber. These results indicated that VIP-PEG-liposomes were capable of chemotactic inhibition of neutrophil migration in response to fmlp.

The present invention is further illustrated by way of the following examples. Example 1 is a comparative example describing the state of the art which illustrates that incorporation of a bioactive VIP peptide into liposomes increases the duration and magnitude of the peptide activity when administered to hamsters with spontaneous hypertension. Example 2 relates to an examination of the same biologically active peptide in association with a sterically stabilized liposome (SSL) according to the methods of invention but in which the liposome provides an even more dramatic increase in peptide activity. Example 3 provides an alternative method for preparing an SSL according to the invention wherein differing preparative techniques are shown to result in vastly different levels of peptide activity. Example 4 provides an analysis of morphological features in liposomes prepared by the methods described in Example 3. Example 5 relates to a modified method for producing SSL with a bioactive peptide wherein simplification of the preparative process does not affect peptide activity in vivo. Example 6 describes manufacture and use of diagnostic liposome products for use in acoustic reflective imaging based on echo-reflective properties of the liposomes. Example 7 relates to the ability of DSPE-PEG5000 to interact with and stabilize interleukin-2 (IL-2) in aqueous medium. Example 8 provides an analysis of the physiochemical properties of sterically stabilized micelles prepared with distearoyl-phosphatidylethanolamine (DSPE) conjugated to different molecular weight (2000, 3000, 5000) PEG. Example 9 studied DSPE conjugated with 1, 2, 3 or 5 kDa PEG in solution, alone or mixed with EYPC by static and dynamic light scattering. Example 10 addresses the issue of covalent conjugation of VIP to SSL and provides an analysis of the targeting ability of VIP-SSL to tumors using MNU-induced rat breast cancer tissues.

### Example 1

### Bioactivity of Peptides in Conventional Liposomes

### (Comparative Example)

According to this example, prior art methods for incorporation of VIP into liposomes were reproduced in order to provide a basis for comparison of the methods of the invention. Because previous observations have suggested that VIP plays a role in regulating vasomotor tone, it was first decided to examine VIP activity in situ on peripheral microcirculation as a function of the vehicle used to dissolve and deliver the peptide. More specifically, a first examination was carried out to determine whether topical administration of VIP could elicit vasodilation in peripheral microcirculation of hamsters with spontaneous hypertension and whether encapsulation of VIP into conventional unilamellar liposomes could modulate any observed response.

Adult male hamsters with spontaneous hypertension (n=21) and age- and genetically-matched normotensive controls (n=20) were purchased from the Canadian Hybrid Farms, Halls Harbor, NS, Canada. In preparation, the animals were anesthetized interperitoneally with sodium pentobarbital (6 mg/100 g body weight) and a tracheostomy was performed to facilitate spontaneous breathing. The left femoral vein was cannulated to inject supplemental anesthesia (2 to 4 mg per 100 g body weight per hour) during the experiment. A catheter was inserted into the left femoral artery to record systemic arterial pressure and heart rate. Body temperature was monitored to maintain a constant 37-38° C. throughout the experiment using a heating pad.

In order to visualize microcirculation of the cheek pouch, previously 20 described methods were employed [Gao, et al ., Life Sci. 64: PL274-PL252 (1994);

Mayhan and Joyner, Microvasc. Res. 28: 159-179 (1984); Mayban and Rubinstein, Biochem. Biophys. Res. Commun. 184:1372-1377 (1992); Raud, Acta Physiol. Scand. Suppl. 578:1-58 (1989); Rubinstein and Mayhan, J. Lab. Clin. Med. 125:313-318 (1995); Rubinstein, et al., Am. J. Physiol. 261 (Heart Circ. Physiol. 30):111913-111918 (1991); and Suzuki, et al., Life Sci. 57:1451-1457 (1995)]. Briefly, the left cheek pouch was spread over a small plastic baseplate, and an incision was made in the outer skin to expose the cheek pouch membrane. The avascular connective tissue layer was removed, and a plastic chamber was positioned over the baseplate and secured in place by suturing the skin around the upper chamber. This arrangement formed a triple-layered complex: the baseplate, the upper chamber, and the cheek pouch membrane exposed between the two plates. The upper chamber was connected to a reservoir containing warmed bicarbonate buffer (37-38° C.) that allowed continuous suffusion of the cheek pouch. The buffer was bubbled continuously with 95% N₂-5% CO₂ (pH 7.4). The chamber was also connected via a three-way valve to an infusion pump (Sage Instruments, Cambridge, Mass.) that allowed controlled administration of drugs into the suffusate. This method of animal preparation was similarly utilized in later investigations as indicated below.

Liposomes containing VIP were prepared according to the methods of Gao, et al., Life Sci. 64: PL274-PL252 (1994); Gregoriadis and Florence, Drugs 45:15-28 (1993); MacDonald, et al., Biochem. Biophys. Acta 1061:297-303 (1991); and Suzuki, et al., Life Sci. 57:1451-1457 (1995). Briefly, a lipid composition consisting of egg yolk phosphatidylcholine (Sigma, St. Louis, Mo.), egg yolk phosphatidylglycerol (Sigma), and cholesterol (Sigma) at a 4:1:5 molar ratio (total phospholipid content, 5 mg) was mixed in chloroform (Sigma) and the solvent evaporated to dryness. The dried lipid film was resuspended in 100 μl 0.15 M NaCl solution containing 0.7 mg VIP by vortex mixing and sonication. The suspension was subjected to five cycles of freeze-thawing using a dry ice-ethanol bath and extruded nine times through two polycarbonate filters (pore size 3 μm; Nuclepore, Pleasanton, Calif.) using a LiposoFast apparatus (capacity of syringe, 0.5 ml; Avestin, Ottawa, ON, Canada). Liposomes were collected using a disposable gel filtration column (Econo-pac IODG, polyacrylamide gel, 10 ml bed vol.) in 0.15 N NaCl [MacDonald, et al., Biochim. Biophys. Acta 1061:297-303 (1991) ]; the liposome fraction was recovered in the void volume and stored at 4° C. until use.

Change in arteriolar diameter was determined as follows. Microcirculation in the cheek pouch was epi-illuminated with a fiber-optic light source and observed through a Nikon microscope. The image was projected through the microscope and into a closed-circuit television system that consisted of low-light television camera, television monitor, and videotape recorder (Panasonic, Yokohama, Japan) . The inner wall diameter of second-order arterioles in the cheek pouch was measured from the video display of the microscope image using a videomicrometer (VIA-100, Boeckeler Instruments, Tucson, Ariz.). Calibration of the magnification of the video system was carried out with a microscope stage micrometer to give microvascular dimensions in micrometers. Vessels were chosen for observation on the basis of clarity on the monitor screen and location within the arteriolar branching pattern in the cheek pouch , In each animal, the same arteriolar segment was used to measure changes in inner wall luminal diameter during the experiment. In some studies, animals were used in more than one treatment group once measures of arteriolar diameter from previous interventions returned to baseline.

VIP alone or encapsulated in liposomes was suffused for 7 minutes at a concentration of VIP of either 0.05 or 0.1 nmol peptide, and more than 30 minutes elapsed between subsequent applications of the peptide. Changes in arteriolar diameter before, during, and after topical application of VIP were determined as outlined above. The concentrations of VIP used in these experiments were based on previous studies [Gao, et al., Life Sci. 64: PL274-PL252 (1994); Suzuki, et al., Life Sci. 57:1451-1457 (1995)].

Results indicated that suffusion of VIP alone at both concentrations was associated with significant vasodilation in normotensive hamsters with the maximal response observed within 4 minutes of the start of suffusion. Arteriolar diameter returned to baseline within 1 minute after suffusion of VIP was stopped. In contrast, suffusion of VIP alone had no significant effects on arteriolar diameter in hamsters with spontaneous hypertension. This blunted response to VIP in hypertensive animals could not be attributed to nonspecific damage to the endothelium because nitroglycerin, an endothelium independent vasodilator in the cheek pouch [Mayban and Rubinstein, Biochem. Biophys. Res. Commun. 184:1372-1377 (1992); Rubinstein, et al, Am. J. Physiol. 261 (Heart Circ. Physiol. 30):111913-111918 (1991)] elicited vasorelaxation of similar magnitude in both groups.

With suffusion of VIP at the same amounts but encapsulated in liposomes, normotensive animals showed significant, concentration-dependent potentiation and prolongation of vasorelaxant effects in comparison with VIP alone. The maximal response was detected 3 to 4 minutes after suffusion began and significant vasodilation persisted almost 9 minutes after suffusion was stopped. In hamsters with spontaneous hypertension, liposome encapsulated VIP produced a significant vasorelaxant effect of magnitude similar to that observed in the normotensive animals. A maximal effect was detected within 4 minutes from the start of suffusion and significant vasodilation persisted over 3 minutes after suffusion was stopped. Even though encapsulation of VIP in liposomes was able to restore vasorelaxant effects of the peptide in hamsters with spontaneous hypertension to a magnitude similar to that observed in normotensive animals, the duration of effect was significantly shorter.

These results suggested that vasodilation elicited by VIP in peripheral microcirculation of normotensive hamsters is composed of two components; the first regulating the magnitude of the response and the second its duration. While the former was expressed in both aqueous and lipid environments, the latter was observed only when VIP was partitioned into lipid bilayers [Gao, et al., Life Sci. 64: PL274-PL252 (1994); Gregoriadis and Florence, Drugs 45:15-28 (1993); MacDonald, et al., Biochim. Biophys. Acta 1061:297-303 (1991); Musso, et al., Biochemistry 27: 8174-8181 (1988); Noda, et al., Biochim. Biophys. Acta 1191: 324-330 (1994); Robinson, et al., Biopolymers 21:1217-1228 (1982); Soloviev, et al., J. Hypertens. 11:623-627 (1993); Suzuki, et al., Life Sci. 57:1451-1457 (1995)] which may provide an appropriate environment for π-helix formation in VIP molecules [Noda, et al., Biochim. Biophys Acta 1191: 324-330 (1994); Robinson, et al, Biopolymers 21:1217-1228 (1982)]. For reasons that are not entirely clear, the lipid-dependent component of VIP-induced vasodilation in peripheral microcirculation was found to be absent in hamsters with essential hypertension.

### Example 2

### Characterization of Bioactivity

### in Sterically Stabilized Liposomes

Having demonstrated that VIP encapsulation in conventional liposomes restored capacity of the peptide to induce vasodilation in hamsters with spontaneous hypertension, changes in VIP activity when associated with the sterically stabilized liposomes of the invention were examined.

Normotensive animals were prepared essentially as described in Example 1 with the following changes. Adult male golden Syrian hamsters (n=28 ; 120-140 g body weight) were anesthetized with pentobarbital sodium (6 mg/100 g body weight, i.p.) and a femoral vein was cannulated to administer the intravascular tracer, fluorescein isothiocyanate labeled dextran (FITC-dextran dissolved in 1.0 ml saline; molecular mass 70 kDa; 40 mg/100 g body weight and administered over 1 minute) and supplemental anesthesia (2-4 mg/100 g body weight/hour). To visualize changes on microcirculation of the cheek pouch, the procedure described above in Example 1 was employed.

Sterically stabilized liposomes (SSL) were prepared as follows. Egg yolk phosphatidylcholine (Sigma), egg yolk phosphatidylglycerol (Sigma), cholesterol (Sigma) and polyethylene glycol (molecular mass, 1,900) linked to distearoyl-phosphatidylethanolamine (molar ratio, 5:1:3.5:0.5; phospholipid content, 17 mmol) were dissolved and mixed in chloroform [Gao, et al., Life Sci. 54: PL247-PL252 (1994); Lasic and Martin. Stealth Liposomes, CRC Press, Inc.: Boca Raton, Fla., 1995; Suzuki, et al., Am. J. Physiol. 271:H282-H287 (1996)]. The solvent was evaporated at 45° C. in a rotary evaporator under vacuum overnight. The resulting lipid film was rehydrated in 250 ml saline, vortexed, bath-sonicated for 5 minutes, and extruded through stacked polycarbonate filters using the LiposoFast apparatus (consecutive pore sizes: 200, 100, 50 nm; AVESTIN, Inc., Ottawa, ON, Canada). Human VIP (0.4 mg) and trehalose (30 mg), a cryoprotectant, were added to the extruded suspension, which was then frozen in acetone-dry ice bath and lyophilized overnight at −46° C. under constant pressure (Foreseen 6, Labconco, Kansas City, Mo.). Thereafter, the lyophilized “cake” was resuspended in 250 ml deionized water. VIP associated with SSL was separated from free VIP by column chromatography (Bio-Gel A-5m, Bio-Rad Laboratories, Richmond, Calif.) and stored at 4° C. for a maximum of 15 days. The size of SSL was 250±50 nm as determined by quasi elastic light scattering (Nicomp model 270 submicron particle sizer, Pacific Scientific, Menlo Park, Calif.). The phospholipid concentration in SSL was determined by the Barlett inorganic phosphate assay [Kates, M. Techniques in Lipidology, Work and Work (Eds.) Elsevier: New York, N.Y. (1972) pp. 354-356]. VIP concentration in SSL was determined by a commercially-available ELISA assay kit (Peninsula Laboratories, Belmont, Calif.) after dissolving SSL with sodium dodecyl sulfate 1%. The recovery was 30% for VIP and 50% for phospholipids, giving a ratio of 0.004 mole VIP/mole of phospholipids.

Determination of arteriolar diameter was carried out as described above in Example 1. In a first group of animals, 0.42 and 0.85 nmol VIP in SSL were suffused for 1 hour in an arbitrary order. At least 45 minutes elapsed between subsequent suffusions of VIP in SSL [Suzuki, et al, Life Sci. 57:1451-1457 (1995); Suzuki, et al., Am. J. Physiol. 271:H282-H287 (1996)]. Arteriolar diameter was measured immediately before suffusion, every minute during suffusion of VIP in SSL and at 5 minute intervals thereafter. Previous observations indicated that suffusion of saline alone for the entire duration of the experiment was associated with no significant change in arteriolar diameter. In another group of animals, VIP in SSL (0.1 nmol) or empty SSL at a concentration equivalent to that in 0.1 nmol VIP in SSL (18 nmol/ml phospholipids) were suffused for 7 minutes.

Suffusion of animals in the first group with 0.42 nmol and 0.85 nmol VIP in SSL for 1 hour produced a significant, concentration dependent, and prolonged increase in arteriolar diameter. Significant vasodilation was observed within 2 minutes of the start of suffusion which maximal within 5 minutes of the beginning of suffusion. Arteriolar diameter returned to baseline levels 50 minutes after suffusion of VIP in SSL was stopped. Suffusion with empty SSL for 1 hour had no significant effect on arteriolar diameter.

Suffusion of normotensive animals in the second group with 0.1 nmol VIP in SSL also elicited a significant increase in arteriolar diameter from baseline but to a lessor extent than that observed in first group. Arteriolar diameter returned to baseline 13 minutes after suffusion of VIP in SSL was stopped. Suffusion of empty SSL had no significant effects on arteriolar diameter. Even though vasodilation for 1 hour was greater than that observed for 7 minute suffusion, the results indicated that using 0.1 nmol peptide would still produce a significant change over baseline.

In order to determine whether the vasodilating effects of VIP in SSL were caused in part by non-specific damage to microvessels resulting in macromolecular efflux from the cheek pouch [Gao, et al., Life Sci. 54: PL247-PL252 (1994); Raud, Acta Physiol. Scand. Suppl. 578:1-58 (1989)], two indices were used to determine clearance of macromolecules from the cheek pouch under control and experimental conditions as previously described [Gao, et al., Life Sci. 54: PL247-PL252 (1994); Raud, Acta Physiol. Scand. Suppl. 578:1-58 (1989)]. The first was a determination of the number of fluorescent “spots” or leaky sites around postcapillary venules and the second was a determination of FITC-dextran clearance from the cheek pouch.

After suffusing animals with bicarbonate buffer for a 30 minute equilibration period, FITC-dextran was administered intravenously. VIP in SSL (0.1 nmol) was then suffused for 7 minutes and the number of leaky sites was determined initially every minute for 7 minutes, and then at 5 minute intervals for 60 minutes thereafter. Clearance of FITC-dextran was determined before suffusion of VIP in SSL and every 5 minutes during and after suffusion for 60 minutes [Gao, et al, Life Sci. 54: PL247-PL252 (1994)].

Results indicated that suffusion of nmol VIP in SSL was not associated with visible leaky site formation. Likewise, clearance of FITC-dextran during suffusion of saline was essentially identical to clearance during suffusion of VIP in SSL.

Combined these results indicated that suffusion of VIP in SSL onto hamster cheek pouch elicits significant and prolonged concentration-dependent vasodilation. This response was not related to non-specific damage to microvascular endothelium because arteriolar diameter returned to baseline once suffusion of VIP in SSL was stopped and because VIP in SSL did not elicit macromolecular efflux from post-capillary venules in the cheek pouch. These results suggested that VIP in SSL could be useful in restoring vascular reactivity in the peripheral microcirculation in certain diseases where endothelium-dependent vasodilation is impaired, such as hypertension, congestive heart failure, diabetes mellitus and impotence [Paul and Ebadi, Neurochem. Int. 23:197-214 (1993); Suzuki, et al., Am. J. Physiol. 271:H282-H287 (1996)].

### Example 3

### Comparison of Bioactivity as

### a Function of Liposome Preparation

Having demonstrated that VIP in SSL exhibits enhanced bioactivity over VIP preparations in conventional liposomes, alternative methods of preparation were examined in order to determine optimal compositions, methods of their preparation, and to further characterize the bioactivity of VIP in SSL.

Two different methods of liposome preparation methods were utilized. In both, the lipids distearoyl-phophatidylethanolamine (PEG-DSPE) (Sequus Pharmaceuticals, Menlo Park, Calif.), Egg yolk phosphatidylcholine (PC) (Sigma Chemical Co., St. Louis, Mo.), and egg yolk phosphatidylglycerol (PG) (Sigma Chemical Co., St. Louis, Mo.), were combined with cholesterol (Sigma Chemical Co., St. Louis, Mo.) at a PEG-DSPE:PC:PG:Chol molar ratio of 0.5:5:1:3.5. Total phospholipid content of the mixture was 17 pmol. The mixture was mixed in chloroform in a round bottom flask, the solvent evaporated at 45° C. in a rotary evaporator (Labconco, Kansas City, Mo.) and the mixture desiccated under vacuum overnight.

In a first method of preparation (not contemplated by the invention), VIP was initially mixed with a lipid composition followed by extrusion and repeated freezing and thawing to produce liposomes. Briefly, the dry lipid film was rehydrated with 250 μl 0.15 M saline (0.9% w/w NaCl) containing 0.4 mg VIP (American Peptide Co., Sunnyvale, Calif.). The mixture was vortexed, sonicated for 5 minutes in a 175.5W water bath sonicator (Fisher Scientific, Itasca, Ill.), and freeze-thawed five times in an acetone-dry ice bath. The suspension was extruded through polycarbonate filters using the Liposofast apparatus (pore size 200 nm, AVESTIN, Inc., Ottawa, ON, Canada). The liposome-associated VIP was separated from the free VIP by column chromatography (BioGel A-5m, Bio-Rad Laboratories, Richmond, Calif.) and stored at 4° C. until use. Column elution was carried out using the 15 M saline solution described above. Vesicle size was determined by quasi elastic light scattering [Alkan-Onyuksel, et al., J. Pharm. Sci. In press (1996)] with a Nicomp 270 particle sizer (Particle Sizing Systems, Santa Barbara, Calif.) and liposomes prepared by this method were found to have an average mean diameter of 224±36 nm.

In a second method of preparation which is contemplated by the invention, a lipid mixture was first extruded, after which VIP was mixed with the formed liposomes. Briefly, a dry lipid film prepared as before was rehydrated with 250 ml 0.15 M saline without VIP. The mixture was vortexed, bath-sonicated for 5 minutes, and extruded through stacked polycarbonate filters of 200, 100, and 50 nm pore size to give a vesicle size of about 80 nm. VIP (0.4 mg) and trehalose (30 mg) (Sigrna Chemical Co., St. Louis, Mo.) as a cryoprotectant were added in powder form to the extruded suspension. The mixture was incubated either at room temperature for two hours or overnight at 4° C., frozen in an acetone-dry ice bath, and lyophilized at -46° C. under a pressure of approximately 5×10⁻³ MBar overnight (Labconco “Freezone 6”, Kansas City, Mo.). The lyophilized “cake” was resuspended with 250 μl deionized water. During freeze-drying, VIP and phospholipid bilayers were in close contact which provides a promotes passive drug loading. Column separation and storage conditions were the same as above. Liposomes prepared by this method were found to have an average diameter of 250±50 run by the method described above, suggesting that freeze-drying permitted vesicle fusion. VIP concentration in the liposomes was determined after treatment with sodium dodecyl sulfate 1% by a VIP ELISA assay kit (Peninsula Laboratories, Belmont, Calif.) and the phospholipid concentration was evaluated by the Barlett inorganic phosphate assay [M. Kates. Techniques in Lipidology, Work and Work (Eds), Elsevier, N.Y. (1972) pp. 354-356]. For both methods of preparation, approximately 30% of the starting VIP was found to be liposome associated and approximately 50% of the starting phospholipids was recovered giving a ratio of approximately 0.004 mole VIP/mole of phospholipid.

Two types of in vivo experiments were performed to determine the vasorelaxant and hypotensive effects of VIP in liposomes prepared by the two methods. In a first series of experiments, the bioactivity of VIP in the liposome preparations was examined as a function of vasodilation, while in the second series of experiments, the duration and efficacy of VIP in the two liposome preparations on mean arterial pressure was measured.

In the first experiments, the bioactivity of VIP in the liposome preparations was measured as a function of change in arteriolar diameter in hamster cheek pouch. Adult male golden Syrian hamsters (n=9) (Sasco, Omaha, Nebr.) were prepared as previously described [Suzuki, et al., Life Sci. 57(15):1451-1457 (1995); Suzuki, et al., Am. J. Physiol. 271:11282-H287 (1996); Suzuki, et al., Am. J. Physiol. In press (1996)] and anesthetized with pentobarbital sodium (2-4 mg/100 g body weight) via a cannulated femoral vein. A femoral artery was cannulated to record systemic arterial pressure and heart rate using a transducer and a strip-chart recorder (Model 260, Gould Instrument Systems Inc., Valley View, Ohio). The visualization of the microcirculation of the cheek pouch, an established animal model to investigate the vasoactive effects of neuropeptides in situ , was conducted as previously described [Suzuki, et al., Life Sci. 57(15):1451-1457 (1995); Suzuki, et al., Am. J. Physiol. 271:11282-H287 (1996); Suzuki, et al., Am. J. Physiol. In press (1996)]. The inner-wall diameter of second order arterioles in the hamster cheek pouch was measured from the video display of the microscope image using a videomicrometer (VIA 100; Boeckeler Instruments, Tucson, Ariz.). In each animal, the same arteriolar segment was used to measure changes in diameter during the experiment. The hamster cheek pouch was first suffused with bicarbonate buffer during a 30 minutes equilibration period, and then with 1.4 ml of each liposome preparations described above for 7 minute.

VIP in liposomes prepared by the first method, outside the scope of the invention, did not elicit an increase in arteriolar diameter significantly different from previously reported observations with 0.1 nmol VIP dissolved in saline, i . e. approximately 10% [Suzuki, et al., Life Sci. 57(15):1451-1457 (1995)]. When this observation is compared to the previous observation that VIP in conventional liposomes prepared with the same method but without an extrusion step shown enhanced and prolonged effects in situ [Suzuki, et al., Life Sci. 57(15):1451-1457 (1995)], three possibilities are suggested to account for the loss of activity of VIP in SSL prepared by the present method; the extrusion process, the lipid composition or the smaller size of the vesicles. Regardless of the reason than SSL prepared by this method did elicit an enhanced or prolonged effect on arteriolar diameter, this result is significant in demonstrating that SSL in general are not amenable to the present invention. VIP (0.1 nmol) in liposomes prepared by the second method and within the scope of the invention, elicited a significant increase in arteriolar diameter from baseline values and the increase persisted for 9 to 16 minutes after suffusion was stopped. This result was more similar to previous observations using conventional liposomes [Suzuki, et al., Life Sci. 57(15):1451-1457 (1995)].

In examining the duration and efficacy of VIP in the two liposome preparations on mean arterial pressure, the following procedure was carried out. Adult mate hamsters with spontaneous hypertension (n=12) were obtained from the Canadian Hybrid Farms (Hall Harbour, Nova Scotia, Canada). Approximately 500 μl each of three test preparations, liposomes prepared by the second method above, VIP in aqueous solution, and liposomes without VIP, were injected administered over the course of 1 minute in the femoral vein. Continuous anesthesia of the animals limited the duration of the experiment to 6 hours.

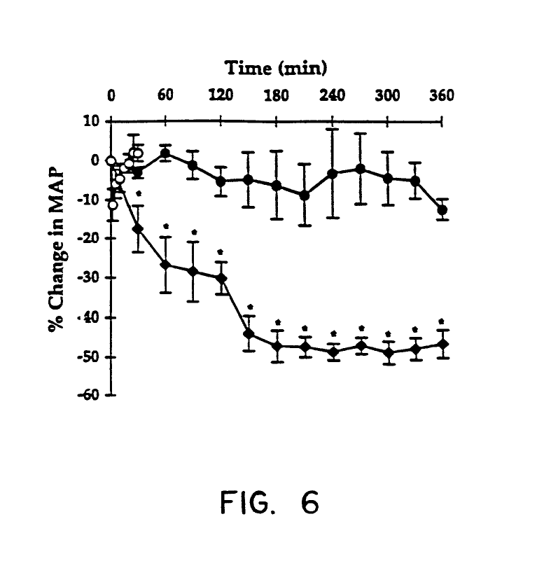

After injection of 0.1 nmol liposome-associated VIP, a significant and gradual decrease in mean arterial pressure up to 50% was observed in the first 2.5 hours which persisted for the 6 hour observation period of the experiment as shown in FIG. 6. No significant effect on mean arterial pressure was observed using empty liposomes or VIP in aqueous solution. These data suggest that intravenously administered VIP in SSL successfully normalized the mean arterial pressure of hamsters with spontaneous hypertension for at least 6 hours. Interestingly, the dose required to produce normal blood pressure was very low compared to previous observations wherein the same amount of VIP in conventional liposomes produced a 30% decrease in mean arterial pressure of normotensive hamsters [Gao, et al., Life Sci. 54:PL247-PL252 (1994)], but this observation may be attributed to a higher sensitivity of hamsters with spontaneous hypertension to VIP.

Since SSL having the same composition and size prepared by the method of the invention (i.e., the second method) retained the VIP activity, the results suggest that extrusion was responsible for the loss of bioactivity in the first liposome preparation. This possibility is consistent with a previous demonstration wherein interleukin-2 was shown to lose more than 25% activity after extrusion [Kedar, et al., J. Immunother. 16:47-59 (1994)], but inconsistent with an observation that vasopressin was not significantly affected by extrusion [Woodle, et al., Pharm Res. 9(2):260-265 (1992)].

### Example 4

### Morphological Evaluation of SSL

For morphological evaluation of vesicle prepared by both methods described in Example 3, liposomes were prepared for freeze-fracture according to standard techniques as reported previously [Alkan-Onyuksel, et al., J. Pharm. Sci. In press (1996)]. Briefly, drops of each liposome suspension were frozen in liquid-nitrogen cooled Freon 22, fractured using a Balzers BAF 301 freeze-etch unit at −115° C., and coated with platinum and carbon. The replicas were cleansed in a minimum of two changes of sodium hypochlorite, washed with distilled water, dried, collected on 200 mesh copper grids, examined and photographed with a JEOL 100CX transmission electron microscope at 80 kv.

Examination of SSL prepared by the method of the invention revealed multivesicular vesicles, suggesting that freeze-drying caused some fusion of the small pre-extruded SSL to form vesicle in a vesicle structures, consistent with the observed increase in mean diameter from 80 nm to 250 nm. This observation is consistent with previously reported fusion events during the freeze-drying/reconstitution process of SSL. [Szucs and Tilcock, Nucl. Med. Biol. 22:263-268 (1995)]. Possibly, the formation of larger vesicles may have promoted the entrapment of VIP molecules inside the final liposomes, while retaining a rather small mean size and distribution required for long circulation times.

### Example 5

### Peptide Activity in a Simplified Liposome Preparation

According to this example a simple method for producing SSL associated with a biologically active peptide is provided which acts to maintain the resulting liposomes at a size approximately less than 200 nm. In addition an alternative method of preparation was examined and the effects of the preparative method on peptide activity determined.

Egg yolk PC, egg yolk PG, cholesterol, and PEG-DSPE were mixed in chloroform at a molar ratio of 5:1:3.5:0.5 and the solvent evaporated using a water bath at 45° C. The lipid film was dried overnight and resuspended in 250 μl saline. The mixture was vortexed, sonicated for 5 minutes and extruded through stacked polycarbonate filters using a LiposoFast apparatus. Human VIP was added to the resulting liposomes having an average diameter of less than 300 nm and the mixture incubated overnight at 4° C. Free VIP was separated from the VIP-associated liposomes using a Bio-gel A-5m column and collected liposomes stored under argon at 4° C. until use. Size of the liposomes determined by quasi electric light scattering indicated an average diameter of 162±59 nm. Phospholipid concentration and VIP recovery were determined as described above and found to be 44% for VIP and 50% for phospholipid, giving a VIP:phospholipid molar ratio of 0.006.

Adult male golden Syrian hypertensive hamsters were prepared for intravital microscopy, cheek pouch microcirculation observed and measured, and mean arterial pressure determined, each technique as described above. Measurements were made with administration of VIP in aqueous solution, VIP in SSL as prepared above, and SSL in the absence of VIP.

Suffusion of VIP in SSL for 7 minutes was associated with a significant, concentration dependent and prolonged increase in arteriolar diameter. Significant vasodilation was observed within 1 minute from the start of suffusion and was maximal within the first 5 minutes. Arteriolar diameter returned to normal levels within 8 minutes after suffusion was stopped. VIP in aqueous solution and empty SSL has no effects.

VIP in SSL also elicited a significant reduction in mean arterial pressure with the maximal effect observed within 30 minutes from the onset of suffusion. Blood pressure remained low during the entire course of the 6 hour observation period. As before, VIP in aqueous solution and empty SSL had no effect.

These results indicated that the dehydration /rehydration step described in Example 3 is not necessary to formation of active liposome preparations. More importantly, liposomes prepared by this method retained an average diameter of less than 200 nm and retained equal, if not higher, VIP activity than either liposome preparation described in Example 3. As an additional advantage, the VIP:phospholipid ratio which resulted from this preparative method was higher (0.006 vs. 0.004) when compared to the method of Example 3.

### Example 6

### SSL in Acoustic Reflectivity Assays

SSL including VIP were prepared and utilized for imaging using acoustic reflectivity measurements as follows.

Liposomes prepared as described in Example 3 were transferred to liquid scintillation vials and imaged with a 20 MHz high-frequency intravascular ultrasound (IVUS) imaging catheter (Boston Scientific Inc., Sunnyvale, Calif.). The IVUS catheter was passed through the vial cap and secured. Instrument settings for gain, zoom, compression, and rejection levels were optimized at the initiation of the experiment and held constant for all samples. Images were recorded onto ½ inch VHS videotape in real time for subsequent playback and image analysis.

Relative echogenicity (apparent brightness) of liposome formulations was objectively assessed by computer-assisted videodensitometry. The process involved acquisition, pre-processing, automated liposome identification, and gray scale quantification. Image processing and analysis were performed with Image Pro Plus Software (Ver. 1.0, Media Cybernetics, Silver Springs, Md.) running on a dedicated computer (486 CPU, 66 MHz). Randomly selected IVUS images were acquired from video tape for each liposome formulation. Images were digitized to 640×480 pixels spatial resolution (approximately 0.045 mm/pixel) and 8 bit (256 levels) amplitude resolution. all analyzed IVUS data were collected at a fixed instrument gain level. The distribution of gray scale values within the image was then adjusted to cover the entire range of possible gray levels using a linear transformation algorithm (i.e., dynamic range was maximized). Image brightness was subjectively scaled such that a reference feature, common to each image, retained a constant gray scale value over all images. An automated-liposome detection routine was then run to identify liposomes suspended in solution within an annular region of interest set at a constant radial distance from the imaging catheter. The automated liposome detection routine identified all “bright” objects within the analysis annulus having a gray scale level greater than 29, a roundness ratio (i.e., ratio of maximum diameter:minimum diameter) less than 2.5, and a size greater than 4 pixels. This procedure excluded virtually all imaging artifacts from the detection algorithm. Thus, object identified were considered to be “liposomes.” Each liposome was outlined and numbered by the computer program. The average gray scale and size of each value of all pixels identified as “liposomes” with a given image was then computed and used to characterize the echogenicity of a given liposome formulation. The results of these experiments demonstrate that the acoustic reflectance of the VIP liposome preparation has a gray scale of 119 (on a gray scale of 0 to 255 with 255 as pure white and 0 as pure black). Larger liposomes produced using lyophilization methods described in PCT Publication WO 93/20802 are characterized by an acoustic reflectance of about 110-120 while liposomes comprising contrast media such as Albunex® have an acoustic reflectance of about 110-120. Accordingly, the invention provides small diameter liposomes while retaining their acoustic imaging properties.

### Example 7

### DSPE-PEG 5000 Increases Physical Stability of Human

### Interleukin-2 In Vitro

According to this example, the ability of DSPE-PEG 5000 to interact with and stabilize IL-2 in aqueous medium was assessed. Protein stability was determined by circular dichroism and fluorescence spectroscopy for secondary and tertiary structure determinations, respectively, turbidity by UV, and visual testing.

IL-2 is a well characterized hydrophobic protein containing a single tryptophan within a four α-helical bundle. These properties render IL-2 ideal for interacting with phospholipids and characterization by fluorescence spectroscopy in that the tertiary structure may be monitored by a shift in the emission wavelength. The isoelectric point (pI) of IL-2 is 7.05. At this pH the protein is chemically most stable but physically least stable. IL-2 was stored in the presence of DSPE-PEG 5000 at the pI of the cytokine so that the protein would be unfolded and electrically neutral to provide a physically interactive environment.

In order to determine the ability of DSPE-PEG 5000 to interact with and stabilize IL-2 in aqueous medium samples were prepared as follows. To obtain the protein in the native state, pure lyophilized recombinant human IL-2 (no excipients) was dissolved in 15 mM sodium acetate at pH 5.0. DSPE-PEG 5000 micelles (100 μM) were prepared by adding 100 mM Tris buffer at pH 7.1, to dry DSPE-PEG 5000. The phospholipid mixture was vortexed for 2 minutes and then sonicated under vacuum for 5 minutes. Micellar size ( ˜25 nm) was assessed in a Nicomp 380 Particle Size Analyzer prior to the addition of protein. Protein was added to the micellar solution or to Tris buffer alone. The final concentration of IL-2 in all protein samples was 0.12 mg/ml. DSPE-PEG 5000 was 70 μM in all DSPE-PEG 5000 samples. Final pH of the solution was between 7.0 and 7.1. DSPE-PEG 5000 in buffer and buffer alone were included as controls. Samples were stored in type I, glass vials with Fluoro Tec® coated stoppers and stored at 5° C. and 25° C. for 28 days. Experiments were carried out in duplicate.

Sample analysis was conducted by circular dichroism (CD) for changes in secondary structure, fluorescence spectroscopy (excitation 295 nm, emission 305-500nm) for changes in tertiary structure, UV (A360) for turbidity, and visual appearance (color, clarity and precipitate). CD spectra were analyzed by SELCON (Softsec version 1.2, 1996) to determine % α-helical content.

Visual turbidity was noted upon initial reconstitution of the lyophilized protein. However, the turbidity observed in the protein solution decreased upon addition into DSPE-PEG 5000 as compared to similar dilution with buffer alone. 100 μM DSPE-PEG 5000 micelles in 100 mM Tris buffer (pH 7.1) yielded a clear, colorless solution. The turbidity observed in the IL-2/DSPE-PEG 5000 samples at 25° C. increased at the same rate as that observed in the DSPE-PEG 5000/buffer samples, suggesting that the increased turbidity was caused primarily by degradation of DSPE-PEG 5000. IL-2/DSPE-PEG 5000 samples stored at 5° C. remained unchanged over the 28-day period studied

Secondary structure of IL-2 was preserved in the presence of DSPE -PEG 5000 for the entire study whereas IL-2 in buffer alone retained <50% of the original α-helical structure after 7 days in solution regardless of storage temperature. No peak shift in fluorescence was observed between IL-2/DSPE-PEG samples and IL-2/buffer samples. However, fluorescence intensity of IL-2/DSPE-PEG 5000 samples was significantly greater than IL-2/buffer samples. The fluorescence from DSPE-PEG 5000 in buffer alone does not explain this difference. The difference in fluorescence intensity is likely due to the greater amount of aggregate and precipitate present in IL-2/buffer samples. A significant amount of precipitate was noted by visual appearance in the IL-2 /buffer samples after 3 days storage.

Results indicated that IL-2 interacts with DSPE-PEG 5000 (molar ratio W˜9:1) at the pI of the protein. This interaction at pH 7 increases the physical stability of IL-2. These results suggested that relatively safe, pegylated phospholipids can be used to stabilize IL-2 in aqueous medium for at least 28 days at 5° C. The underlying mechanism of interaction remains unclear.

### Example 8

### Effect of PEG Chain Length on Size, CMC and Solubilization Potential of

### Sterically-stabilized Phospholipid Micelles

According to this example, micelle compositions of the invention were further characterized. Particularly, the physiochemical properties of sterically stabilized micelles prepared with DSPE conjugated to molecular weight 2000, 3000, and 5000 PEG were analyzed. The critical micelle concentration (CMC) of phospholipids was determined at pH 7.4 and 25° C. using a water-insoluble fluorescent probe (1,6-diphenyl-1,3,5-hexatriene). Micellar size was determined by quasi-elastic light scattering. Solubilization potential of micelles was determined using diazepam as a model hydrophobic drug and RP-HPLC.

As a result, CMC of DSPE-PEG micelles increased from 0.5 to 1.5 μM range as molecular weight of PEG increased from 2000 to 5000 Mean hydrodynamic diameters (±SEM) of micelles were 16.8±0.3, 20.3±0.6 and 23.9±2.1 nm for DSPE-PEG 2000, 3000, and 5000, respectively. Furthermore, maximal concentration (±SD) of diazepam solubilized in DSPE-PEG 200, 3000, and 5000 was 288.97±7.51, 224.26±6.22 and 195.92±19.73 μg/ml at a constant concentration of phospholipid (1 mM), respectively.

These results indicated that shorter PEG chain length of DSPE-PEG results in smaller micellar size and lower CMC with increased solubilization potential for insoluble drugs. This suggests that DSPE-PEG 2000 micelles are better solubilizers for small hydrophobic molecules, which could be related to an increase in the number of micelles/molar lipid concentration.

### Example 9

### Characterization of Phospholipid Micelles

### by Light Scattering Investigations

According to this example, DSPE conjugated with 1, 2, 3 or 5 kDa PEG in solution, alone or mixed with egg yolk phosphatidylcholine (EYPC) were studied by static (SLS) and dynamic light scattering (DLS).

SLS and DLS was used to study micelles in DSPE conjugated with PEG of nominal molecular weight 1, 2, 3 or 5 KDa, either alone or with 25mole % EYPC, as a function of total phospholipid concentration. The phospholipids were dissolved in methanol and dried as a film. The films were dissolved in 10 mM HEPES buffer, pH 7.4, 0.15 NaCl with agitation. The samples were then flushed with nitrogen, sealed and incubated in the dark at room temperature for 48 hours. Samples were passed through a 0.2 μ filter to eliminate dust.

The apparatus was configured to measure SLS and DIS as a function of momentum transfer, Q. Q is related to the scattering angle, 2θ, wavelength, λ=632.8, and medium index of refraction, n, as,

$$Q = \frac{4 π n}{λ} ( sin θ )$$

Correlation functions are measured using ALV-5000 Multiple Tau Digital Correlator over lag times between 2×10⁻⁷ and 10 s. Multiple angle scattering intensity and correlation functions over a large dynamic range allow detailed characterization of micelle size, shape and polydispersity.

The Guinier approximations for SLS of globular particles,

$$I ( Q ) = Δ M exp - \frac{\vert {Q}^{2} {R}_{g}^{2} \vert}{\vert 3 \vert} ,$$

and equivalent forms for rods and sheets (Hjelm et al., J. Phys. Chem., B104:197 (2000), are used to make estimates of the particle radius of gyration, R_{g} in the domain R_{g}Q<1.3 R_{g} and shape. DLS gives estimates of the diffusion coefficient, D, of particles in a media of viscosity η, by measurements of the time-dependent correlation function. D can be used to estimate the particle hydrodynamic radius, R_{H} through the Stokes-Einstein equation,

$${R}_{H} = \frac{k T}{6 πη D}$$

These results indicated that DSPE-PEG 1000 does not form micelles in either simple or mixed surfactant solutions. DSPE-PEG at 2, 3, and 5 KDa formed micelles at 1.1 mM and lower with and without EYPC. With EYPC the micelles were considerably larger. At higher concentrations DSPE-PEG/EYPC mixtures form an anistropic phase. The characterization of particular forms met the expectations that when EYPC is incorporated into the simple DSPE-PEG micelles, the particular curvature and shape will change to give a bigger hydrophobic core and therefore the solubilization potential of phospholipid micelles will improve. The results indicate that the size can be controlled by the addition of a second phospholipid. This shows that the approach may be useful in developing micellar drug delivery systems.

### Example 10

### VIP Receptors as Molecular Targets of Breast Cancer

According to this example the therapeutic uses of the invention are analyzed. Previously, sterically stabilized liposomes (SSL) were prepared with VIP non-covalently associated on their surface. In this example, the need to conjugate VIP covalently to SSL is studied and the targeting ability of VIP-SSL to n-methyl nitrosourea (MNU)-induced rat breast cancer in vitro is tested.

DSPE-PEG₃₄₀₀-NHS [1,2-dioleoyi-sn-glycero-3 -phosphoethanolamine-n- [poly(ethylene glycol)]-N-hydroxy succinamide, PEG M_{w} 3400] and polyethylene glycol (M_{w} 2000) conjugated distearyl phosphatidylethanolamine (DSPE-PEG₂₀₀₀) were obtained from Sheanvater Polymers, Inc. (Huntsville, Ala.). BODIPY-Chol (flourescent cholesterol) was obtained from Molecular Probes Inc. (Portland, Ore.). Fluo-VIP™ (Portland, Ore.). Fluo-VIP ™ fluorescein labeled VIP) was purchased from Advanced Bioconcept (Montreal, Quebec, Canada). VIP (human/rat) was synthesized, using solid-phase synthesis by Protein Research Laboratory at Research Resources Center, University of Illinois at Chicago. Egg-phosphatidylcholine (PC) and cholesterol (CH) were obtained from Sygena (Switzerland). Virgin female Sprague-Dawley rats (˜140 g body weight) were obtained from Harlan (Indianapolis, Ind.).

In conducting research using animals, the investigators adhered to the Institutional Animal Care Committee guidelines and to the Guide for the Care and Use of Laboratory Animals of the Institute of Laboratory Animal Resources, National Research Council.

An activated DSPE-PEG (DSPE-PEG₃₄₀₀ -NHS) was used to conjugate VIP to DSPE-PEG₃₄₀₀. This reaction takes place between amines and NHS group, which acts as the linking agent VIP and DSPE-PEG₃₄₀₀-NHS in the molar ratio of 1:5 (VIP:DSPE-PEG₃₄₀₀ -NHS) were dissolved separately in 0.01 M isotonic HEPES buffer, pH 6.6. DPSE-PEG₃₄₀₀ -NHS solution was added in small increments over 1-2 min to the VIP solution at 4° C. and then stopped by adding glycine solution to the reaction mixture to consume the remaining NHS moieties. The conjugation was tested using SDDS-PAGE and subsequent staining with first Coomassie Blue R-250 and then silver stain. The VIP conjugated to DSPE-PEG₃₄₀₀ (DSPE-PEG₃₄₀₀-VIP) was subsequently used to prepare fluorescent VIP-SSL.

Breast cancer was induced in rats with MNU as previously described in G. O. Udeani et al.,Cancer Research, 57:3424-3428 (1997). Briefly, virgin female Sprague-Dawley rats, 36 days old, weighing ˜140 g, were anesthetized with ketamine/xylazine (13.3/1.3 mg per 100 g body weight, i.m.). Each animal received a single intravenous injection of MNU (50 mg/kg body weight) in acidified saline (pH 5.0), via the tail vein. The rats were weighed weekly. They were palpated every week, starting at 3 weeks post-MNU administration. Palpable mammary tumors were detected within 100-150 days after injection.

For testing the in vitro binding, BODIPY-Chol (a non-exchangeable fluorescent probe) containing liposomes, were prepared with film rehydration-extrusion method, as described in S. Dagar et al., Pharm. Sci., 1:S-294 (1998) and M. Patel et al., Proc. Int. Symp. Control. Rel. Bioact. Mat., 24:913-914 (1997) but incorporated the probe at 1:1500 molar ratio (lipid:probe) in the lipid mixture. Egg phosphatidylcholine (PC), cholesterol (CH), DSPE-PEG₂₀₀₀ and dipalmitoyl phosphatidylglycerol (DPPG) in the molar ratios of PC:DPPG:DSPE-PEG₂₀₀₀:CH of 0.50:0.10:0.03:0.35 were used to form the sterically stabilized liposomes by film rehydration and reconstitution using isotonic, 0.01 M HEPES buffer (pH 6.6). This was followed by extrusion through polycarbonate filters (100 nm) using a Liposofast® (Avestin Inc., Canada) extruder. The size of final liposomes was ˜140 nm as determined using quasi-elastic light scattering (NICOMP 370, Particle Sizing Systems, Santa Barbara, Calif.). DSPE-PEG₃₄₀₀-VIP was inserted into these fluorescent liposomes by overnight incubation at 4° C. to form fluorescent VIP conjugated sterically stabilized liposomes (VIP-SSL).

The rats were euthanized by exposure to carbon dioxide in a closed chamber. Normal and cancerous breast tissue were excised, frozen immediately in liquid nitrogen and stored at −70° C. until use. The frozen breast tissue was cut into 20-mm sections and mounted on microscopic slides.. They were then fixed with 4% formaldehyde and allowed to air-dry for 10 min. Adjacent 5 mm thick frozen tissue sections, were stained with hemotoxylin and eosin to confirm the presence or absence of cancer in the breast tissue. The presence of VIP-R in these rat breast cancer tissues was confirmed using a fluorescent VIP, FluoVIP™ as described in S. Dagar et al., Breast Cancer Res. Treatment (2000) in press. Twenty micormeter sections of MNU-induced rat breast cancer tissues were cut using a cryotome, placed on a slide, fixed with 4% formalin for 20 min., and then air-dried for 10 min. The BODIPY-Chol containing VIP-SSL were added to the sections and incubated for 1 h at room temperature. At the end of the incubation period, the slides were washed with 0.01 M isotonic HEPES buffer, pH 6.6, four times for 60 s each. The slides were then observed with a Zeiss Camera (Carl Zeiss Inc., Thomwood, N.Y.) and photographed. All photographs were taken with a 2 min exposure using Kodak Elite Chrome 400 photographic film. The VIP-SSL were compared to SSL without VIP or with non-covalently associated VIP and the difference in number of fluorescent liposomes present on the tissue indicated the difference in attachment of VIP-SSL to MNU-induced rat breast cancer tissues.

The reaction conditions were optimized after systemic variation of pH, reaction time, reaction temperature, molar ration of VIP: DSPE-PEG₃₄₀₀-NHS and stirring rate. It was found that the conditions of reaction (2 h at 4° C., pH 6.6, gentle stirring and 1:5 molar ratio) currently used gave the best results. Therefore, the subsequent experiments were done using these optimized conditions. The stained gel (SDS-PAGE) of the conjugation mixture showed that most of the product is 1:1 conjugate of VIP and DSPE-PEG₃₄₀₀ (DSPE- PEG₃₄₀₀--VIP), and free VIP and 1:2 conjugate of VIP and DSPE-PEG₃₄₀₀ exist at much lesser extent as compared to 1:1 DSPE-PEG₃₄₀₀-VIP conjugate. Furthermore, the fluorescence microphotographs of breast cancer tissues indicated that more VIP-SSL were attached to MNU-induced rat breast cancer tissue sections while SSL without VIP or with non-covalently associated VIP, showed no significant attachment.

In this experiment VIP was successfully conjugated to DSPE-PEG₃₄₀₀ and incorporated into preformed sterically stabilized liposomes to form a VIP-SSL construct. The results showed the feasibility of this novel construct to actively target to MNU-induced rat breast cancer in vitro.

Numerous modifications and variations in the invention as set forth in the above illustrative examples are expected to occur to those skilled in the art. Consequently only such limitations as appear in the appended claims should be placed on the invention.

What is claimed is:

1. A method of treating a disease state selected from the group consisting of autism, multiple sclerosis, eneuresis, Parkinson's disease, amyotrophic lateral sclerosis, brain ischemia, stroke, Cerebral palsy sleep disorder, feeding disorder and AIDS-associated dementias, comprising the step of administering to an individual suffering from the disease state an amount of a liposome composition effective to alleviate conditions associated with the disease state, said liposome composition prepared by a method comprising the steps of:
a) mixing a combination of lipids wherein said combination includes at least one lipid component covalently bonded to a water-soluble polymer;
b) forming sterically stabilized liposomes from said combination of lipids;
c) obtaining liposomes having an average diameter of less than about 300 nm; and
d) incubating liposomes from step (c) with a biologically active amphipathic compound under conditions in which said compound becomes associated with said liposomes from step (c) in an active conformation, wherein at least one amphipathic compound is a member of the VIP/glucagon/secretin family of peptides including peptide fragments and analogs.

2. The method according to claim 1 wherein said liposome composition comprises unilamellar liposomes.

3. The method according to claim 1 wherein said liposome composition comprise multivesicular liposomes.

4. The method of according to claim 3 wherein said multivesicular liposomes are produced by carrying out the steps of sequentially dehydrating and rehydrating liposomes obtained in step (c) with said biologically active peptide.

5. The method according to any one of claims 1 through 4 wherein said water-soluble polymer is polyethylene glycol (PEG).

6. The method according to claim 1 wherein the amphipathic compound is characterized by having one or more α- or π-helical domains in its biologically active conformation.

7. The method according to claim 6 wherein the amphipathic compound is a member of the vasoactive intestinal peptide (VIP)/growth hormone releasing factor (GRF) family of peptides.

8. The method according to claim 7 wherein the amphipathic compound is a member of the VIP/glucagon/secretin family of peptides, including peptide fragments and analogs thereof.

9. The method according to claim 1 wherein the liposomes obtained in step (c) have an average diameter or less than about 200 nm.

10. The method according to claim 9 wherein the liposomes obtained in step (c) have an average diameter or less than about 100 nm.

11. The method according to any one of claims 1, 8, or 9 wherein the liposomes are obtained in step (c) by extrusion to form liposomes having a selected average diameter.

12. The method according to any one of claims 1, 8, or 9 wherein the liposomes are obtained in step (c) by size selection.

13. The method according to claim 1 wherein the combination of lipids consists of distearoyl-phosphatidylethanolamine covalently bonded to PEG (PEG-DSPE), phosphatidylcholine (PC), and phosphatidylglycerol (PG) in further combination cholesterol (Chol).

14. The method according to claim 13 wherein the combination of lipids are combined with cholesterol in a PEG-DSPE:PC:PG:Chol molar ratio of 0.5:5:1:3.5.
